(19) United States
(12) Patent Application Publication
(10) Pub. No.: US 2023/0122620 A1
(43) Pub. Date: Apr. 20, 2023

(54) PROCESS FOR MAKING HYDROXYLATED CYCLOPENTYLPYRIMIDINE COMPOUNDS

(71) Applicant: F. HOFFMANN LA-ROCHE AG, Little Falls, NJ (US)

(72) Inventors: Dainis Kaldre, Basel (CH); Joerg Sedelmeier, Basel (CH)

(21) Appl. No.: 18/079,729

(22) Filed: Dec. 12, 2022

Related U.S. Application Data
(63) Continuation of application No. PCT/EP2021/065873, filed on Jun. 14, 2021.

(30) Foreign Application Priority Data
Jun. 16, 2020 (EP) 20180148.7

(51) Int. Cl. C07D 239/70; B01J 31/12

(52) U.S. Cl. CPC C07D 239/70; B01J 31/122; B01J 2231/4233; B01J 2531/22

(57) ABSTRACT

Provided herein is a process for synthesis of intermediates for ipatasertib related to large scale manufacture of (R)-5-methyl-4-(piperazin-1-yl)-5,6-dihydro-7H-cyclopenta[d]pyrimidin-7-one.

### CROSS REFERENCE TO RELATED APPLICATIONS

This application claims the benefit of and priority to EP Application EP20180148.7, filed Jun. 16, 2020, the content of which is incorporated herein by reference in its entirety.

### FIELD OF THE INVENTION

Provided herein is a process for synthesis of intermediates for Ipatasertib related to large scale manufacture of (R)-5-methyl-4-(piperazin-1-yl)-5,6-dihydro-7H-cyclopenta[d]pyrimidin-7-one and N-protected derivatives thereof.

### BACKGROUND

AKT (also known as Protein Kinase B) is a serine/threonine protein kinase that is overexpressed in certain human tumors. Ipatasertib is an AKT inhibitor that is currently being evaluated in clinical trials for the treatment of solid tumors, gastric cancer, and prostate cancer. Ipatasertib is disclosed in, for example, U.S. Pat. No. 8,063,050 (see, e.g., Example 14), as well as International Patent Application Publication No. WO 2008/006040.

(R)-5-methyl-4-(piperazin-1-yl)-5,6-dihydro-7H-cyclopenta[d]pyrimidin-7-one, or the N-protected derivative thereof, is used as an intermediate in the synthesis of Ipatasertib. Various different processes for preparing this intermediate are reported, for example in, International Patent Application Publication No. WO 2013/173736, International Patent Application Publication No. WO 2013/173768, and International Patent Application Publication No. WO 2016/049414.

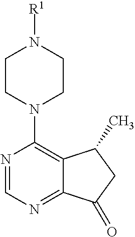

(R)-5-methyl-4-(piperazin-1-yl)-5,6-dihydro-7H-cyclopenta[d]pyrimidin-7-one, or the N-protected derivative thereof, wherein R¹ is hydrogen or an amino protecting group

However, it is known that scale-up of chemical processes can result in unexpected conditions such as, for example, increased impurities or decreased yields. Accordingly, there is a need for improved processes for the synthesis of Ipatasertib that increase yields and/or decrease impurities.

### SUMMARY

Provided herein are solutions to the problems above and other problems in the art.

Provided herein is a process for preparing (R)-5-methyl-4-(piperazin-1-yl)-5,6-dihydro-7H-cyclopenta[d]pyrimidin-7-one (compound of formula (1) wherein R¹ is hydrogen) wherein the process comprises a continuous flow reaction. Also provided is a process for preparing N-protected derivatives thereof (compound of formula (1) wherein R¹ is an amino protecting group).

In one embodiment the continuous flow reaction mentioned above comprises cyclizing a compound of formula (II), or pharmaceutically acceptable salt thereof, to provide a compound of formula (I), or pharmaceutically acceptable salt thereof:

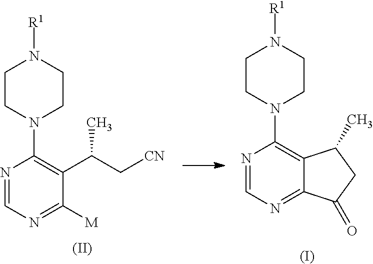

wherein:
R¹ is hydrogen or an amino protecting group; and
M is Li or MgX, wherein X is Br or I.

In certain embodiments, R¹ is an amino protecting group.

In one embodiment the continuous flow reaction described above comprises cyclizing a compound of formula (II)

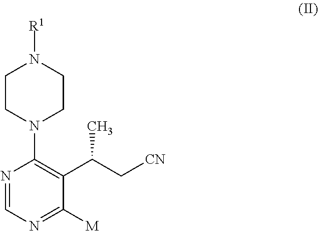

or a pharmaceutically acceptable salt thereof, wherein:
R¹ is hydrogen or an amino protecting group; and
M is Li or MgX, wherein X is Br or I.

In certain embodiments, R¹ is an amino protecting group.

In one embodiment the continuous flow reaction described above comprises cyclizing a compound of formula (II)

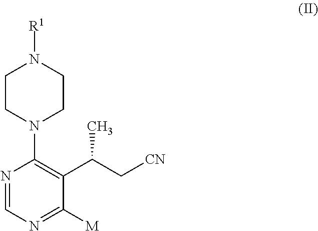

or a pharmaceutically acceptable salt thereof, wherein:
R¹ is hydrogen or an amino protecting group; and
M is MgX, wherein X is Br or I.

In certain embodiments, R¹ is an amino protecting group.

In certain embodiments, M is MgBr. In certain embodiments, R¹ is an amino protecting group and M is MgBr.

For example, in certain embodiments, the continuous flow reaction described above comprises cyclizing a compound of formula (II):

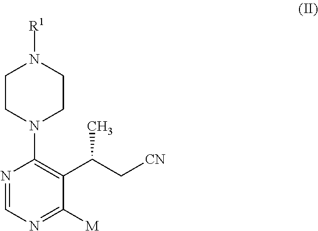

or a pharmaceutically acceptable salt thereof, wherein:
R¹ an amino protecting group; and
M is MgBr.

In one embodiment, the continuous flow reaction comprises iPrMgBr as metalating reagent.

The iPrMgBr permits continuous mode processing with a very fast reaction, reduced levels of dimerization by-products (amongst others), and easily isolated final products.

In one aspect provided herein is a process as exemplified by FIG. 1.

In another aspect provided herein is a process as exemplified by FIG. 2.

In a third aspect provided herein is a process for the preparation of a compound of formula (I) as described herein or a pharmaceutically acceptable salt thereof the process comprising a continuous flow reaction wherein in step (a) the compound of formula (III) or a pharmaceutically acceptable salt thereof, wherein R¹ is as defined above, and R³ is iodo or bromo, in a coordinating ethereal solvent or a non-coordinating apolar solvent, or a mixture of such solvents, is reacted with iPrMgBr in MeTHF to form a solid Mg-imine complex and wherein in step (b) an aqueous acid salt is added to the mixture obtained from step (a), thereby forming the compound of formula (I) or a pharmaceutically acceptable salt thereof.

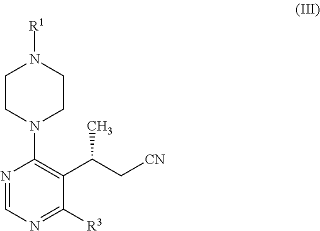

The processes described herein has one or more of the following advantages:
(i) improved yields by 10-20%,
(ii) precipitation of a transient reaction intermediate protects it from further decomposition,
(iii) improved process robustness and better controllability,
(iv) better purity,
(v) no additional re-crystallization required to meet specification,
(vi) minimal scale up factors and short hold times for production, and
(vii) significant cost savings in the scaled up process per ton of Ipatasertib.

The present embodiments can be understood more fully by reference to the detailed description and examples, which are intended to exemplify non-limiting embodiments.

### BRIEF DESCRIPTION OF THE FIGURES

FIG. 1 depicts one diagram of the process described herein showing continuous flow reaction of compound 4 to compound 1 (or a pharmaceutically acceptable salt thereof). Arrows indicate flow between continuous stirred tank reactors (CSTRs). Flow rates are determined as described herein.

FIG. 2 depicts one diagram of the process described herein showing continuous flow reaction of compound 4 to compound 1 (or a pharmaceutically acceptable salt thereof). Arrows indicate flow between continuous stirred tank reactors (CSTRs). Flow rates are determined as described herein.

### DETAILED DESCRIPTION

Unless defined otherwise, all technical and scientific terms used herein have the same meaning as commonly understood by those of ordinary skill in the art. See, e.g., Singleton et al., DICTIONARY OF MICROBIOLOGY AND MOLECULAR BIOLOGY 2nd ed., J. Wiley & Sons (New York, N.Y. 1994); Sambrook et al., MOLECULAR CLONING, A LABORATORY MANUAL, Cold Springs Harbor Press (Cold Springs Harbor, N Y 1989). Any methods, devices and materials similar or equivalent to those described herein can be used in the practice of the present disclosure.

Provided herein are processes for synthesis of a compound of formula (I) or a pharmaceutically acceptable salt thereof as described herein, using a Li or Mg metalating agent, such as iPrMgBr. The methods described herein possess advantages over previously described methods in that they allow for continuous mode operation and avoid the need for additional purification steps, including re-crystallization. The reaction with iPrMgBr in MeTHF described herein results in a solid Mg-imine complex in suspension during early process steps. Compared to other processes, this surprising result contributes to increased yields and enhanced purity profiles by mitigating consecutive over-reactions to form undesired impurities. In some example, scaled-up reactions using the process described herein increase the yield of the compound of formula (I) or a pharmaceutically acceptable salt thereof by about 10-20% compared to other processes described herein.

Reference will now be made in detail to certain embodiments of which are illustrated in the accompanying structures and formulas. It will be understood that these embodiments are not intended to limit the present disclosure to those embodiments. On the contrary, the present disclosure intended to cover all alternatives, modifications, and equivalents which may be included within the scope of the present disclosure.

One skilled in the art will recognize many methods and materials similar or equivalent to those described herein, which could be used in the practice the present disclosure. The present disclosure is in no way limited to the methods and materials described. In the event that one or more of the incorporated literature and similar materials differs from or contradicts this application, including but not limited to defined terms, term usage, described techniques, or the like, this application controls.

The term “pharmaceutically acceptable salts” denotes salts which are not biologically or otherwise undesirable. Pharmaceutically acceptable salts include both acid and base addition salts. The phrase “pharmaceutically acceptable” indicates that the substance or composition must be compatible chemically and/or toxicologically, with the other ingredients comprising a formulation, and/or the mammal being treated therewith.

The term “pharmaceutically acceptable acid addition salt” denotes those pharmaceutically acceptable salts formed with inorganic acids such as hydrochloric acid, hydrobromic acid, sulfuric acid, nitric acid, carbonic acid, phosphoric acid, and organic acids selected from aliphatic, cycloaliphatic, aromatic, araliphatic, heterocyclic, carboxylic, and sulfonic classes of organic acids such as formic acid, acetic acid, propionic acid, glycolic acid, gluconic acid, lactic acid, pyruvic acid, oxalic acid, malic acid, maleic acid, malonic acid, succinic acid, fumaric acid, tartaric acid, citric acid, aspartic acid, ascorbic acid, glutamic acid, anthranilic acid, benzoic acid, cinnamic acid, mandelic acid, embonic acid, phenylacetic acid, methanesulfonic acid “mesylate”, ethanesulfonic acid, p-toluenesulfonic acid, and salicyclic acid.

The term “pharmaceutically acceptable base addition salt” denotes those pharmaceutically acceptable salts formed with an organic or inorganic base. Examples of acceptable inorganic bases include sodium, potassium, ammonium, calcium, magnesium, iron, zinc, copper, manganese, and aluminum salts. Salts derived from pharmaceutically acceptable organic nontoxic bases includes salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-diethylaminoethanol, trimethamine, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, and polyamine resins.

The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%.

“Amino protecting group” are groups described in detail in Protecting Groups in Organic Synthesis, T. W. Greene and P. G. M. Wuts, 3^{rd} edition, John Wiley & Sons, 1999, and protect an amino functional group in a given chemical reaction. Exemplary amino protecting groups include, but are not limited to, (i) amide R(C═O)— groups, such as formyl, acetyl, chloroacetyl, trichloroacetyl, trifluoroacetyl, and phenylacetyl; (ii) carbamate RO(C═O)— groups, wherein R is methyl, ethyl, 9-fluorenylmethyl (Fmoc), 2,2,2-trichloroethyl (Troc), 2-trimethylsilylethyl (Teoc), Cert-butyl (Boc), and benzyl (Cbz); (iii) sulfonamide R—(SO₂)— groups, wherein R is toluene, benzene, methyl, trifluoromethyl, and 2-nitrobenene; (iv) R—CH₂— groups wherein R is a benzene radical, toluene radical, paramethoxybenzene radical (PMB), or 2-(trimethylsilyl)ethoxy (SEM), and (v) (R)₃C— groups, wherein R is a benzene radical (trityl). In one embodiment, the amino protecting group is acetyl (CH₃C═O)—, Ac), trifluoroacetyl (CF₃C═O)—), benzyl (PhCH₂—, Bn), triphenylmethyl ((Ph)₃C—, trityl), p-toluenesulfonyl (pCH₃-Ph-SO₂—), p-methoxybenzyl (pCH₃O-Ph-CH₂—, PMB), tert-butyloxycarbonyl (tBuOC(═O)—, Boc), 9-fluorenylmethyloxycarbonyl (9-fluorenylmethyl-C(═O), and carbobenzyloxy (PhCH₂OC(═O)—), some of which are considered labile under acidic conditions. In one embodiment, the amino protecting group is acetyl (CH₃C═O)—, Ac), trifluoroacetyl (CF₃C═O)—, triphenylmethyl ((Ph)₃C—, trityl), p-toluenesulfonyl (pCH₃-Ph-SO₂—), p-methoxybenzyl (pCH₃O-Ph-CH₂—, PMB), tert-butyloxycarbonyl (tBuOC(═O)—, Boc), 9-fluorenylmethyloxycarbonyl (9-fluorenylmethyl-C(═O), and carbobenzyloxy (PhCH₂OC(═O)—), each of which are considered labile under acidic conditions. In one preferred embodiment, the amino protecting group is tert-butyloxycarbonyl (Boc).

“Continuous flow reaction” is used herein to mean a chemical reaction which is run in a continuously flowing stream rather than in batch production. In other words, pumps move fluid into a flow system, wherein the fluids contact one another. If these fluids are reactive, a reaction takes place. In some embodiments, microreactors are used. In some embodiments, tubular or plug flow reactors are used. In some other embodiments, continuous stirred tank reactors (CSTR) are used.

In one embodiment, provided is a process for the preparation of a compound of formula (I) or a pharmaceutically acceptable salt thereof,

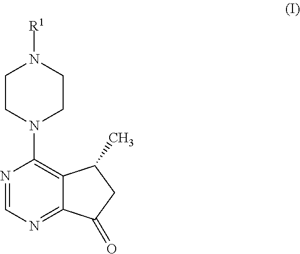

wherein the process comprises a continuous flow reaction, with R¹ of the compound of formula (I) is hydrogen or an amino protecting group.

In one embodiment of this application, the continuous flow reaction comprises cyclization of a compound of formula (II)

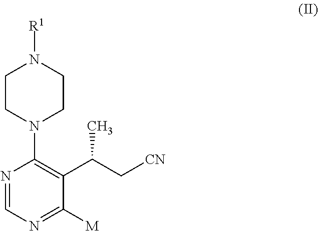

or a pharmaceutically acceptable salt thereof,
wherein:
R¹ is hydrogen or an amino protecting group; and
M is Li or Mg.

In certain embodiments, R¹ is an amino protecting group.

In one embodiment, the continuous flow reaction described above comprises cyclizing a compound of formula (II)

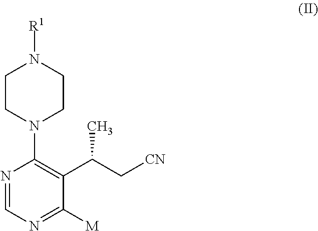

or a pharmaceutically acceptable salt thereof, wherein:
R¹ is hydrogen or an amino protecting group; and
M is Li or MgX, wherein X is Br or I.

In one embodiment M is Mg in the form of MgX.

In certain embodiments, R¹ is an amino protecting group.

In one embodiment, the continuous flow reaction described above comprises cyclizing a compound of formula (II)

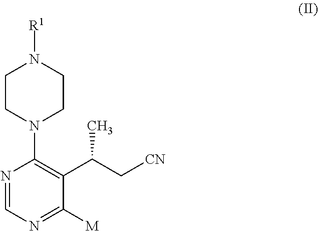

or a pharmaceutically acceptable salt thereof, wherein:
R¹ is hydrogen or an amino protecting group; and M is MgX, wherein X is Br or I.

In certain embodiments, R¹ is an amino protecting group.

In certain embodiments, X is Br.

For example, in a preferred embodiment, the continuous flow reaction described above comprises cyclizing a compound of formula (II)

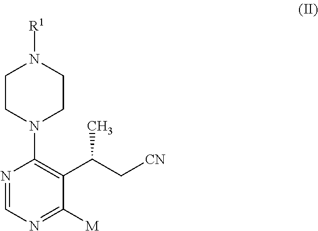

or a pharmaceutically acceptable salt thereof, wherein:
R¹ is an amino protecting group; and
M is MgBr.

In one embodiment, the continuous flow reaction comprises:

(a) contacting a compound of formula (III), or a pharmaceutically acceptable salt thereof

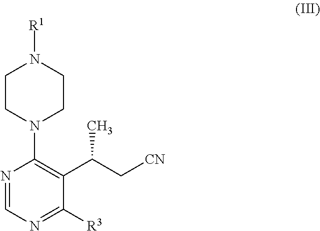

wherein:
R¹ is hydrogen or an amino protecting group,
R³ is iodo or bromo; and
   with a Li or Mg metalating agent to form a compound of formula (I).

In certain embodiments, R¹ is an amino protecting group.

In one embodiment, the compound of formula (III), wherein R³ is bromo, is a compound of formula (IV) or a pharmaceutically acceptable salt thereof:

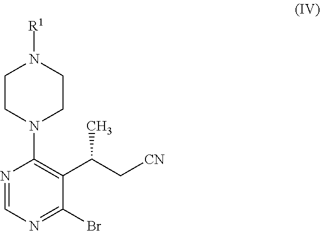

wherein R¹ is hydrogen or an amino protecting group.

In one preferred embodiment, the compound of formula (III) is a compound of formula (IV) or a pharmaceutically acceptable salt thereof

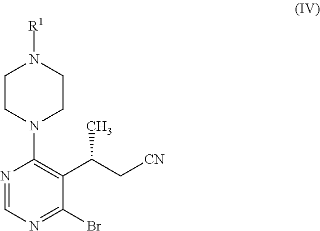

wherein R¹ is an amino protecting group.

In one embodiment, the metalating agent is iPrMgBr.

In one embodiment, the amino protecting group is acetyl, trifluoroacetyl, phthalimidyl, benzyl, triphenylmethyl, benzylidenyl, p-toluenesulfonyl, p-methoxybenzyl, tert-butyloxycarbonyl, 9-fluorenylmethyloxycarbonyl or carbobenzyloxy. In one embodiment, the amino protecting group is acetyl, trifluoroacetyl, benzyl, triphenylmethyl, p-toluenesulfonyl, p-methoxybenzyl, tert-butyloxycarbonyl, 9-fluorenylmethyloxycarbonyl or carbobenzyloxy. In one preferred embodiment, the amino protecting group is tert-butyloxycarbonyl.

In another embodiment, R¹ is hydrogen.

In one embodiment, the non-coordinating apolar solvent is hexane, heptane, toluene, xylene, or a mixture thereof. In one embodiment, the non-coordinating apolar solvent is toluene.

In one embodiment, the coordinating ethereal solvent is diethyl ether (EtOEt), tert-butyl methyl ether (MeOtBu), diisopropyl ether (iPrOiPr), dioxane, cPentylOMe, tetrahydrofuran (THF), or methyl tetrahydrofuran (MeTHF), or a mixture thereof. In one embodiment, the coordinating ethereal solvent is diethyl ether. In another embodiment, the coordinating ethereal solvent is MeTHF.

In one embodiment, the Li and Mg metalating agent is selected from the group consisting of iPrMgCl, iPrMgCl*LiCl, iPrMgBr, iPrMgI, tBuMgCl, sBuMgCl, sBuMgCl*LiCl, nBuLi, sBuLi, nHexLi.

In one preferred embodiment, the Li and Mg metalating agent is iPrMgBr. In one further embodiment, the iPrMgBr is in MeTHF.

In one embodiment, the compound of formula (III) is in EtOEt, MeOtBu, iPrOiPr, dioxane, cPentylOMe, THF, MeTHF, hexane, heptane, toluene, or xylene, or a mixture thereof. In a preferred embodiment, the compound of formula (III) is in a mixture comprising MeTHF and toluene.

In one embodiment of the process described herein, iPrMgBr is added to the compound of formula (III) by multi-dosing in one or more portions using continuous stirred tank reactors (CSTRs). Where iPrMgBr is added to the compound of formula (III), the addition can occur over one or more steps (e.g. a first reaction step of the compound of formula (III) with iPrMgBr and a second reaction step of the compound formula (III) with an additional amount of iPrMgBr). The heat produced during the Grignard reaction can be high and separation or sequential addition of iPrMgBr can reduce any undesired effects of excess heat. In addition, when added sequentially, two or more additions of iPrMgBr can reduce the effective local concentration of iPrMgBr, promoting reaction efficiency and reducing undesired byproduct formation, including but not limited to, dimerization of the compound of formula (III) with the compound of formula (I).

In one preferred embodiment, the compound of formula (IV) is in a mixture comprising MeTHF and toluene.

In one embodiment of the process described herein, iPrMgBr is added to the compound of formula (IV) in one or more steps. Where iPrMgBr is added to the compound of formula (IV), the addition can occur over one or more steps (e.g. a first reaction step of the compound of formula (IV) with iPrMgBr and a second reaction step of the compound formula (IV) with an additional amount of iPrMgBr). The heat produced during the Grignard reaction can be high and separation or sequential addition of iPrMgBr can reduce any undesired effects of excess heat. In addition, when added sequentially, two or more additions of iPrMgBr can reduce the effective local concentration of iPrMgBr, promoting reaction efficiency and reducing undesired byproduct formation, including but not limited to, dimerization of the compound of formula (IV) with the compound of formula (I).

In one embodiment, in step (a) the compound of formula (III) is transferred to the compound of formula (II) and the compound of formula (II) is transferred to a Mg-imine complex and R¹ is hydrogen or an amino protecting group and wherein in step (b) an aqueous acid salt is added to the mixture obtained from step (a), thereby forming the compound of formula (I).

In one embodiment, the Mg-imine complex is formed as a solid in suspension and R¹ is hydrogen or an amino protecting group. In one embodiment, R¹ is an amino protecting group. In one embodiment, R¹ is tert-butyloxycarbonyl.

In one embodiment, the Mg-imine complex and is solid in suspension until reaction with the acid salt. The solid is transferred in suspension by the overall continuous process flow. The solid is transferred in suspension between CSTRs by the overall process flow.

In one embodiment, the Mg-imine complex is selected from the group shown below

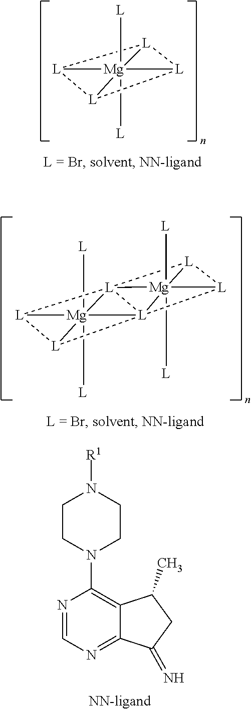

wherein n is 1 to infinity. In one more embodiment n is in a range, to form a monomer, dimer, trimer, oligomer or polymer complex.

In one preferred embodiment, the Mg-imine complex is selected from the group shown below:

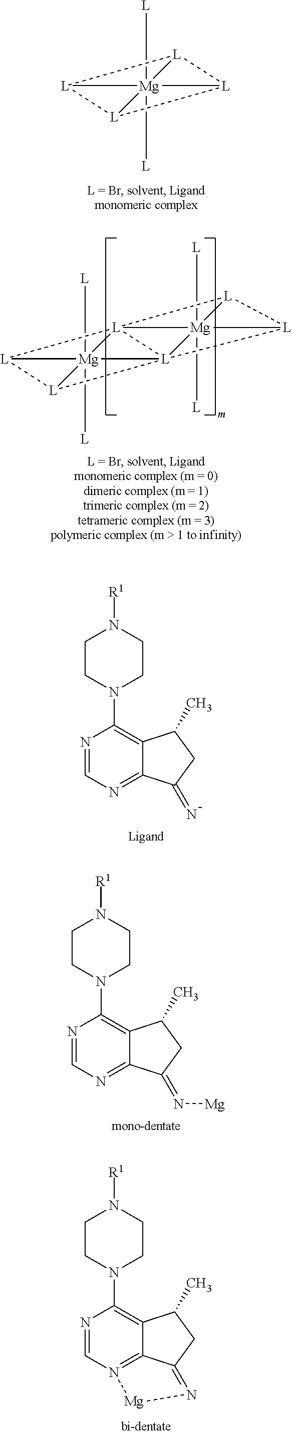

wherein m is 0 to infinity.

In one embodiment, m is in the range of 0 to 100, 0 to 200, 0 to 300, 0 to 400, 0 to 500, 0 to 1000, 0 to 5000, or 0 to 10000. In certain embodiments, the complex is a monomer (wherein m is 0). In certain embodiments, the complex is a dimer (wherein m is 1). In certain embodiments, the complex is a tetramer (wherein m is 3). In certain embodiments, the complex is polymeric, e.g., wherein m>1 to infinity, e.g., m is in the range of 2 to 100, 2 to 200, 2 to 300, 2 to 400, 2 to 500, 2 to 1000, 2 to 5000, or 2 to 10000. In certain embodiments, the solid in suspension comprises a mixture of Mg-imine complexes.

The ligand L is selected from the group consisting of Br, any solvent used (e.g. a coordinating ethereal solvent) in the reaction, the NN-ligand shown above, and any combination thereof. In another embodiment, the Mg-imine complex comprises a mixture of different Mg-imine complexes.

As Mg-imine complex has an ionic character, it forms a solid in suspension (in the apolar solvent mixture MeTHF and toluene) within the continuous flow reaction. Under the conditions of the present invention the Mg-imine complex precipitates and is transported as a solid with the continuous flow.

In one embodiment, the molar ratio of iPrMgBr to the compound of formula (III) is about 1:1 to about 1.5:1. In another embodiment, the molar ratio of iPrMgBr to the compound of formula (III) is about 1.3:1. In still another embodiment, the molar ratio of iPrMgBr to the compound of formula (III) is about 1:1.

An one embodiment, the molar ratio of iPrMgBr to the compound of formula (IV) is about 1:1 to about 1.5:1. In another embodiment, the molar ratio of iPrMgBr to the compound of formula (IV) is about 1.3:1. In still another embodiment, the molar ratio of iPrMgBr to the compound of formula (IV) is about 1:1.

Without being bound by any particular theory, the solid Mg-imine complex in suspension reacts slower than when in solution thereby reducing undesirable, consecutive byproduct formation and decreased yields. Thus, the processes described herein increase purity of the compound of formula (I) and increase the overall yield when compared to other processes. Furthermore, in certain embodiments described herein, the process does not require a re-crystallization step to yield the compound of formula (I) due to the more selective and cleaner reaction.

In one embodiment, the aqueous acid salt is HCl, H₂SO₄, NaHSO₄, H₃PO₄, NaH₂PO₄, Na₂HPO₄, Na₂HPO₄, NaH₂citrate, NH₄Cl, or oxalic acid, or a mixture thereof.

Aqueous means water or any other solvent comprising water.

In one embodiment, the acid salt is aqueous HCl, H₂SO₄, NaHSO₄, H₃PO₄, NaH₂PO₄, Na₂HPO₄, Na₂HPO₄, NaH₂citrate, NH₄Cl, or oxalic acid, or a mixture thereof.

In one embodiment, the aqueous acid salt is aqueous H₂SO₄ or NaHSO₄. In another embodiment, the acid salt is aqueous NaH₂PO₄, Na₂HPO₄, or Na₂HPO₄.

In one embodiment, the acid salt is aqueous NaHSO₄.

The processes described herein can be performed in one or more continuous stirred tank reactors (CSTR). In such embodiments, the process is performed in 1, 2, 3, 4, 5, 6, or 7 CSTRs. In one embodiment, the process is performed in at least 5 CSTRs. In another embodiment, the process if performed in 5 CSTRs.

Where the process is performed using CSTRs, in one embodiment, is a first CSTR comprising a solution of the compound of formula (III) in a organic solvent, the organic solvent comprising one or more of EtOEt, MeOtBu, iPrOiPr, dioxane, cPentylOMe, THF, MeTHF, hexane, heptane, toluene, or xylene, or a mixture thereof. In one embodiment, the mixture comprises MeTHF. In another embodiment, the mixture comprises toluene. In another embodiment, the compound of formula (III) is in a mixture of MeTHF and toluene.

In one such embodiment, the process includes adding to the solution of the compound of formula (III) iPrMgBr in MeTHF. The molar ratio of iPrMgBr to the compound of formula (III) in the first CSTR is as described herein. In one embodiment, the molar ratio of iPrMgBr to the compound of formula (III) in the first CSTR is about 1:1. In one embodiment, the residence time for the first CSTR is about 5 min to about 30 min. In one embodiment, the residence time for the first CSTR is about 15 min. In one embodiment the compound of formula (III) is a compound of formula (IV).

The term “residence time” refers to the residence time distribution (RTD) of a continuous flow system and is a probability distribution function that describes the amount of time a molecule or compound could spend inside the reactor setup.

Where the reaction of the compound of formula (III) and iPrMgBr is performed in the first CSTR, the process can further comprise a second CSTR. In one embodiment, 1.3 equivalents of iPrMgBr in MeTHF are added in one or two steps. In one embodiment, within a first addition 1.0 equivalents of iPrMgBr in MeTHF are to the reaction mixture in the first CSTR and allowed to react followed by a second addition of 0.3 equivalents of iPrMgBr in MeTHF.

In one embodiment the reaction comprises a second CSTR comprising a mixture of the compounds of formula (IV) and the Mg-imine complex in a suspension of MeTHF and toluene, wherein to this mixture about 0.3 equivalents of iPrMgBr in MeTHF are added.

In one embodiment, the contents of the first CSTR are transferred to the second CSTR and allowed to react.

In one embodiment, the residence time for the second CSTR is about 5 min to about 30 min. In one embodiment, the residence time for the second CSTR is about 15 min.

In one embodiment, the residence time in the second CSTR is sufficient to form a Mg-imine complex in a suspension of MeTHF and toluene with no remaining compound of formula (III) (e.g. the reaction goes to completion). In such embodiments, where the residence time in the second CSTR is sufficient as described herein, the contents of the second CSTR can be transferred to a fourth CSTR as provided herein (i.e. skipping the third CSTR described herein). The numbering of the CSTRs remains unaltered if the third CSTR is skipped. In one embodiment, the compound of formula (III) is a compound of formula (IV).

In one embodiment wherein the residence time in the second CSTR is not sufficient to form an Mg-imine complex with no remaining compound of formula (III) (e.g. the reaction does not go to completion), the process comprises a third CSTR where the contents of the second CSTR are transferred to the third CSTR and stirred in the third CSTR. In one embodiment, the residence time for the third CSTR is about 5 min to about 30 min. In one embodiment, the residence time for the third CSTR is about 15 min. In one embodiment, reaction with the 1.3 equivalents of iPrMgBr in the third CSTR is performed until completion (e.g. absence of starting material compound formula (III). In one embodiment, the compound of formula (III) is a compound of formula (IV).

In one embodiment, wherein the Mg-imine complex is formed as a solid in suspension by reacting the compound of formula (III) with iPrMgBr and is transferred as a suspension or slurry between CSTRs. In another embodiment, the solid Mg-imine complex is in suspension.

In one embodiment, wherein the Mg-imine complex is formed as a solid in suspension by reacting the compound of formula (IV) with iPrMgBr and is transferred as a suspension between CSTRs. In another embodiment, the Mg-imine complex is in a suspension of MeTHF and toluene.

In one embodiment, the contents of the second or third CSTR are transferred to a fourth CSTR thereby forming a biphasic mixture (emulsion). The fourth CSTR comprises an aqueous acid salt as described herein. In one embodiment, the acid salt is aqueous NaHSO₄. The pH of the biphasic mixture in the fourth CSTR can be adjusted and maintained at a pH of about 1 to about 3. In one embodiment, the pH of the biphasic mixture after addition of the aqueous NaHSO₄ is about 2. In one embodiment, the residence time of the fourth CSTR is about 2 min to about 10 min. In another embodiment, the residence time of the fourth CSTR is about 3 min to about 8 min. In another embodiment, the residence time of the fourth CSTR is about 4 min to about 6 min. In another embodiment, the residence time of the fourth CSTR is about 4 min. In another embodiment, the residence time of the fourth CSTR is about 5 min. In another embodiment, the residence time of the fourth CSTR is about 4.45 min.

Formation of the compound of formula (I) as described above can result in the compound of formula (I) partitioning to the aqueous phase of the mixture in the fourth CSTR. In one embodiment, the nitrogen atoms of the compound of formula (I) are protonated following contact with aqueous NaHSO₄.

In one embodiment, the process comprises a fifth CSTR comprising a base in water. The base is selected from the group consisting of NaOH, KOH, Na₂CO₃, and K₂CO₃. In one embodiment, the base is NaOH.

In another embodiment, the process comprises a fifth CSTR comprising NaOH in water. In one embodiment, the content of the fourth CSTR as an emulsion is transferred to the fifth CSTR thereby forming a biphasic mixture. In one embodiment, the biphasic mixture after addition of NaOH in water has a pH of about 4 to about 7. In one embodiment, the biphasic mixture after addition of NaOH in water has a pH of about 4 to about 5.5. In another embodiment, the pH of the biphasic mixture after addition of NaOH in water is about 4.8±0.2. In another embodiment, the pH of the biphasic mixture after addition of NaOH in water is about 4.6±0.2. In one embodiment, the nitrogen atoms of the compound of formula (I) are de-protonated at a pH of about 5 resulting in partitioning of the compound of formula (I) to the organic phase. In one embodiment, the residence time of the fifth CSTR is about 2 min to about 10 min. In another embodiment, the residence time of the fifth CSTR is about 3 min to about 8 min. In another embodiment, the residence time of the fifth CSTR is about 4 min to about 6 min. In another embodiment, the residence time of the fifth CSTR is about 4 min. In another embodiment, the residence time of the fifth CSTR is about 5 min. In another embodiment, the residence time of the fifth CSTR is about 4.45 min.

The processes described herein can further comprise one or more separation vessels. In one embodiment, the contents of the fifth CSTR are transferred to a first separation vessel, thereby separating the organic and aqueous phases. In one embodiment, the resulting organic phase is: (i) washed with water; (ii) distilled; (iii) precipitated; and (iv) dried to isolate the compound of formula (I) or a pharmaceutically acceptable salt thereof.

The processes described herein can also further comprise a sixth CSTR comprising water. In one embodiment, the resulting organic phase from the separation vessel is added to the sixth CSTR. In another embodiment, the processes described herein further comprise a second separation vessel, whereupon the contents of the sixth CSTR are added to the second separation vessel, thereby separating organic and aqueous phases. In one embodiment, the resulting separated organic phase is: (i) distilled; (ii) precipitated; and (iii) dried to isolate the compound of formula (I) or a pharmaceutically acceptable salt thereof.

In one embodiment, in the compound of formula (I), R¹ is H representing a compound of formula (1):

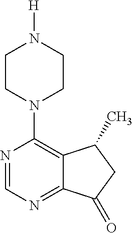 [SMILES: C[C@@H]1CC(=O)c2ncnc(N3CCNCC3)c21]

This is formed by deprotecting the N-protected derivative thereof, wherein R¹ is an amino protecting group. The deprotetion of the compound of formula (I) or any other compound described herein may be achieved by any method known to the person skilled in the art and is usually performed later in the synthesis of Ipatasertib after the continuous flow reaction of this invention.

The compounds described herein may contain asymmetric or chiral centers, and therefore exist in different stereoisomeric forms. It is intended that all stereoisomeric forms of the compounds of the invention, including but not limited to, diastereomers, enantiomers and atropisomers, as well as mixtures thereof such as racemic mixtures, form part of the present invention.

In still another aspect provided herein is a process for the preparation of a compound of formula (I) as described herein, the process comprising:
(a) solubilizing a compound of formula (IV)

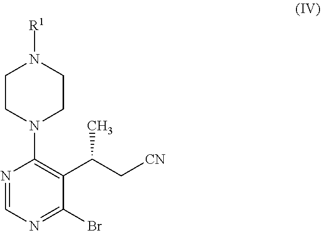

in a mixture of toluene and MeTHF in a first continuous stirred tank reactor (CSTR);
(b) adding iPrMgBr in MeTHF to the solubilized compound of step (a) in the first, thereby forming a Mg-imine complex as a solid;
(c) transferring the contents of the first CSTR to a second CSTR to which iPrMgBr in MeTHF is added, thereby forming additional Mg-imine complex as a solid;
(d) transferring the contents of the second CSTR to a third CSTR to which toluene is added, thereby forming additional Mg-imine complex as a solid;
(e) transferring the contents of the third CSTR to a fourth CSTR to which aqueous NaHSO₄ is added to set a pH about 2, thereby forming the compound of formula (I);
(f) transferring the contents of the fourth CSTR to a fifth CSTR to which a base in water is added to set a pH of about 5; and
(g) transferring the contents of the fifth CSTR to a first separation vessel to separate the compound of formula (I).

In certain embodiments, R¹ is as described herein. In particular embodiments R¹ is an amino protecting group.

In still another aspect provided herein is a process for the preparation of a compound of formula (I) as described herein, the process comprising:
(a) solubilizing a compound of formula (IV)

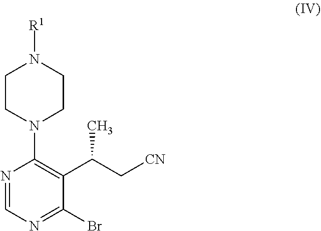

in a mixture of toluene and MeTHF in a first continuous stirred tank reactor (CSTR);
(b) adding iPrMgBr in MeTHF to the solubilized compound of step (a) in the first, thereby forming a Mg-imine complex as a solid in suspension;
(c) transferring the contents of the first CSTR to a second CSTR to which iPrMgBr in MeTHF is added, thereby forming a Mg-imine complex as a solid in suspension;
(d) transferring the contents of the second CSTR to a third CSTR to which toluene is added, thereby forming a Mg-imine complex as a solid in suspension;
(e) transferring the contents of the third CSTR to a fourth CSTR to which aqueous NaHSO₄ is added to set a pH about 2, thereby forming the compound of formula (I);
(f) transferring the contents of the fourth CSTR to a fifth CSTR to which NaOH in water is added to set a pH of about 5; and
(g) transferring the contents of the fifth CSTR to a first separation vessel to separate the compound of formula (I).

In certain embodiments, R¹ is as described herein. In particular embodiments R¹ is an amino protecting group.

In still another aspect provided herein is a process for the preparation of a compound of formula (I) as described herein, the process comprising:
(a) solubilizing a compound of formula (IV)

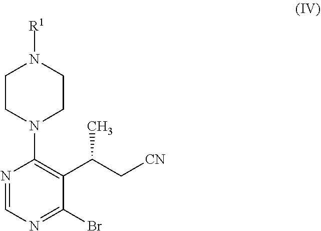

in a mixture of toluene and MeTHF in a first continuous stirred tank reactor (CSTR);
(b) adding iPrMgBr in MeTHF to the solubilized compound of step (a) in the first CSTR and wherein the first CSTR has a residence time of about 15 min, thereby forming a Mg-imine complex as a solid;
(c) transferring the contents of the first CSTR to a second CSTR to which iPrMgBr in MeTHF is added wherein the second CSTR has a residence time of about 15 min, thereby forming a Mg-imine complex as a solid;
(d) transferring the contents of the second CSTR to a third CSTR to which toluene is added wherein the third CSTR has a residence time of about 15 min, thereby forming a Mg-imine complex as a solid;
(e) transferring the contents of the third CSTR to a fourth CSTR to which aqueous NaHSO₄ is added to set a pH about 2, thereby forming the compound of formula (I) and allowing the reaction to proceed for about 4.45 min;
(f) transferring the contents of the fourth CSTR to a fifth CSTR to which a base in water is added to set a pH of about 5 and allowing the reaction to proceed for about 4.45 min; and
(g) transferring the contents of the fifth CSTR to a first separation vessel to separate the compound of formula (I).

In certain embodiments, R¹ is as described herein. In particular embodiments R¹ is an amino protecting group.

In still another aspect provided herein is a process for the preparation of a compound of formula (I) as described herein, the process comprising:
(a) solubilizing a compound of formula (IV)

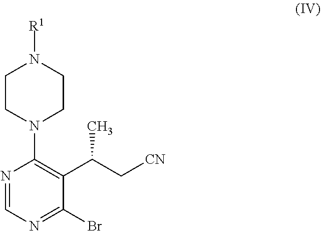

in a mixture of toluene and MeTHF in a first continuous stirred tank reactor (CSTR);
(b) adding iPrMgBr in MeTHF to the solubilized compound of step (a) in the first CSTR and wherein the first CSTR has a residence time of about 15 min, thereby forming a Mg-imine complex as a solid;
(c) transferring the contents of the first CSTR to a second CSTR to which iPrMgBr in MeTHF is added wherein the second CSTR has a residence time of about 15 min, thereby forming additional Mg-imine complex as a solid;
(d) transferring the contents of the second CSTR to a third CSTR to which toluene is added wherein the third CSTR has a residence time of about 15 min, thereby forming additional Mg-imine complex as a solid;
(e) transferring the contents of the third CSTR to a fourth CSTR to which aqueous NaHSO₄ is added to set a pH about 2, thereby forming the compound of formula (I) and allowing the reaction to proceed for about 4.45 min;
(f) transferring the contents of the fourth CSTR to a fifth CSTR to which a base in water is added to set a pH of about 5 and allowing the reaction to proceed for about 4.45 min; and
(g) transferring the contents of the fifth CSTR to a first separation vessel to separate the compound of formula (I).

In certain embodiments, R¹ is as described herein. In particular embodiments R¹ is an amino protecting group.

In still another aspect provided herein is a process for the preparation of a compound of formula (I) as described herein, the process comprising:
(a) solubilizing a compound of formula (IV)

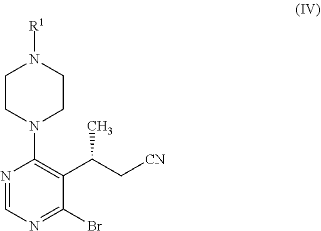

in a mixture of toluene and MeTHF in a first continuous stirred tank reactor (CSTR);
(b) adding iPrMgBr in MeTHF to the solubilized compound of step (a) in the first CSTR and wherein the first CSTR has a residence time of about 15 min, thereby forming a Mg-imine complex as a solid in suspension:
(c) transferring the contents of the first CSTR to a second CSTR to which iPrMgBr in MeTHF is added wherein the second CSTR has a residence time of about 15 min, thereby additional Mg-imine complex as a solid in suspension;
(d) transferring the contents of the second CSTR to a third CSTR to which toluene is added wherein the third CSTR has a residence time of about 15 min, thereby forming additional Mg-imine complex as a solid in suspension in suspension;
(e) transferring the contents of the third CSTR to a fourth CSTR to which aqueous NaHSO₄ is added to set a pH about 2, thereby forming the compound of formula (I) and allowing the reaction to proceed for about 4.45 min;
(f) transferring the contents of the fourth CSTR to a fifth CSTR to which NaOH in water is added to set a pH of about 5 and allowing the reaction to proceed for about 4.45 min; and
(g) transferring the contents of the fifth CSTR to a first separation vessel to separate the compound of formula (I).

In certain embodiments, R¹ is as described herein. In particular embodiments R¹ is an amino protecting group.

In such embodiments of the process above, the conditions such as temperatures, concentrations, transfer rates, reaction times, and volumes are as described herein.

In some embodiments, the process includes online monitoring through NMR, HPLC or IR.

In preferred one embodiment, the deprotection of the compound of formula (I) or any other compound described herein may be achieved by any method known to the person skilled in the art and is performed after the continuous flow reaction. In some preferred embodiments, the compound of formula (I) is used in the following reactions steps as amino protecting form, and the deprotection is performed several reaction steps later.

### Embodiments

Embodiment 1: A process for the preparation of a compound of formula (I) or a pharmaceutically acceptable salt thereof,

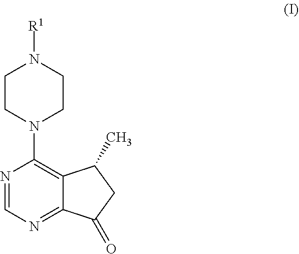

wherein the process comprises a continuous flow reaction, with R¹ of the compound of formula (I) is hydrogen or an amino protecting group.

Embodiment 1A: A process for the preparation of a compound of formula (I) or a pharmaceutically acceptable salt thereof,

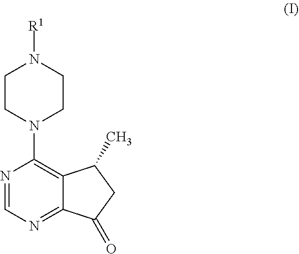

wherein the process comprises a continuous flow reaction, with R¹ of the compound of formula (I) is an amino protecting group.

Embodiment 2: The process of embodiment 1, wherein the continuous flow reaction comprises cyclization of a compound of formula (II)

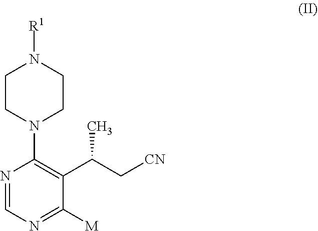

or a pharmaceutically acceptable salt thereof, wherein:
R¹ is hydrogen or an amino protecting group; and
M is Li or Mg.

Embodiment 2A: The process of embodiment 1, wherein the continuous flow reaction comprises cyclization of a compound of formula (II)

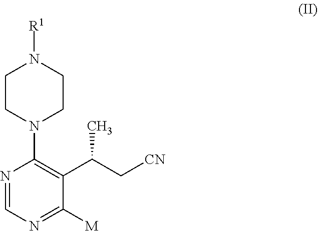

or a pharmaceutically acceptable salt thereof,
wherein:
R¹ is hydrogen or an amino protecting group; and
M is MgX, wherein X is Br or I.

Embodiment 2B: The process of embodiment 1, wherein the continuous flow reaction comprises cyclization of a compound of formula (II)

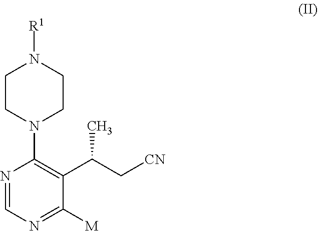

or a pharmaceutically acceptable salt thereof, wherein:
R¹ is an amino protecting group; and
M is MgX, wherein X is Br or I.

Embodiment 2C: The process of embodiment 1, wherein the continuous flow reaction comprises cyclization of a compound of formula (II)

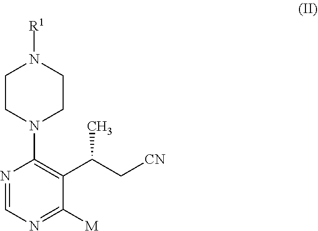

or a pharmaceutically acceptable salt thereof,
wherein:
R¹ is an amino protecting group; and
M is MgX, wherein X is Br.

Embodiment 3: The process of embodiment 2, wherein M is Mg.

Embodiment 4: The process of embodiment 2 or embodiment 3, wherein the continuous flow reaction comprises:
(a) contacting a compound of formula (III) or a pharmaceutically acceptable salt thereof

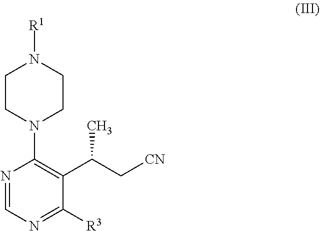

wherein:
R¹ is hydrogen or an amino protecting group; and
R³ is iodo or bromo;
   with a Li or Mg metalating agent to form a compound of formula (I).

Embodiment 4a: The process of embodiment 2 or embodiment 3, wherein the continuous flow reaction comprises:
(a) contacting a compound of formula (III) or a pharmaceutically acceptable salt thereof

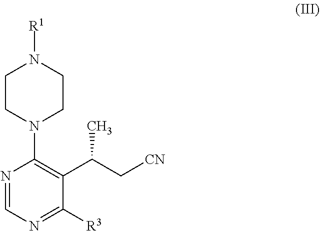

wherein:
R¹ is an amino protecting group; and
R³ is iodo or bromo;
   with a Li or Mg metalating agent to form a compound of formula (I).

Embodiment 5: The process of embodiment 4, wherein the compound of formula (III) is a compound of formula (IV) or a pharmaceutically acceptable salt thereof

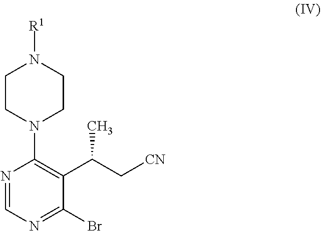

wherein R¹ is hydrogen or an amino protecting group.

Embodiment 5: The process of embodiment 4, wherein the compound of formula (III) is a compound of formula (IV) or a pharmaceutically acceptable salt thereof

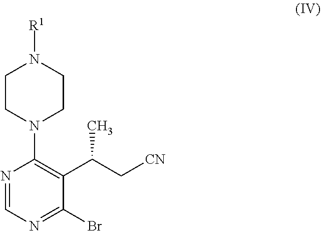

wherein R¹ is an amino protecting group.

Embodiment 6: The process of embodiment 5, wherein the metalating agent is iPrMgBr.

Embodiment 7: The process of any one of embodiments 1-6, wherein said continuous flow reaction is conducted in a coordinating ethereal solvent or a non-coordinating apolar solvent, or a mixture thereof.

Embodiment 8: The process of any one of embodiments 1-7, wherein said continuous flow reaction is conducted in a coordinating ethereal solvent.

Embodiment 9: The process of any one of embodiments 1-7, wherein said continuous flow reaction is conducted in a non-coordinating apolar solvent.

Embodiment 10: The process of embodiment 8, wherein the coordinating ethereal solvent is EtOEt, MeOtBu, iPrOiPr, dioxane, cPentylOMe, THF, or MeTHF.

Embodiment 11: The process of embodiment 9, wherein the non-coordinating apolar solvent is hexane, heptane, toluene, or xylene, or a mixture thereof.

Embodiment 12: The process of any one of embodiments 6-11, wherein the iPrMgBr is in MeTHF.

Embodiment 13: The process of any one of embodiments 5-12, wherein the compound of formula (IV) is in EtOEt, MeOtBu, iPrOiPr, dioxane, cPentylOMe, THF, MeTHF, hexane, heptane, toluene, or xylene, or a mixture thereof.

Embodiment 14: The process of embodiment 13, wherein the compound of formula (IV) is in a mixture comprising MeTHF and toluene.

Embodiment 15: The process of any one of embodiments 6-14, wherein the iPrMgBr is added to the compound of formula (IV).

Embodiment 16: The process of any one of embodiments 4-15, wherein in step (a) the compound of formula (III) is transferred to the compound of formula (II) and the compound of formula (II) is transferred to a Mg-imine complex as a solid and R¹ is hydrogen or an amino protecting group and wherein in step (b) an aqueous acid salt is added to the mixture obtained from step (a), thereby forming the compound of formula (I) or a pharmaceutically acceptable salt thereof.

Embodiment 17: The process of embodiment 16, the Mg-imine complex is formed as a solid in suspension and R¹ is hydrogen or an amino protecting group.

Embodiment 17a: The process of embodiment 17, the Mg-imine complex is formed as a solid in suspension and R¹ is an amino protecting group.

Embodiment 18: The process of any one of embodiments 6-17, wherein the molar ratio of iPrMgBr to compound of formula (IV) is about 1:1 to about 1.5:1.

Embodiment 19: The process of any one of embodiments 6-18, wherein the ratio of iPrMgBr to compound of formula (IV) is about 1.3 to about 1.

Embodiment 20: The process of any one of embodiments 6-19, wherein the ratio of iPrMgBr to compound of formula (IV) is about 1:1.

Embodiment 21: The process of any one of embodiments 6-20, wherein the iPrMgBr is added to the compound of formula (IV) in one or more steps.

Embodiment 22: The process of any one of embodiments 16-21, wherein the aqueous acid salt is selected from the group consisting of acetic acid, HCl, H₂SO₄, NaHSO₄, H₃PO₄, NaH₂PO₄, Na₂HPO₄, NaH₂citrate, NH₄Cl, or oxalic acid, or a mixture thereof.

Embodiment 23: The process of embodiment 22, wherein the aqueous acid salt is NaHSO₄.

Embodiment 24: The process of any one of embodiments 1-23, wherein the compound of formula (I) is optionally (a) washed; (b) distilled; (c) precipitated; and (d) dried.

Embodiment 25: The process of any one of embodiments 1-24, wherein the process does not require a re-crystallization step.

Embodiment 26: The process of any one of embodiments 1-25, wherein the process is performed in one or more continuous stirred tank reactors (CSTR).

Embodiment 27: The process of embodiment 26, wherein the process is performed using 1, 2, 3, 4, 5, 6, or 7 CSTRs.

Embodiment 28: The process of embodiment 26 or embodiment 27, wherein the process is performed using 5 CSTRs.

Embodiment 29: The process of any one of embodiments 26-28, comprising a first CSTR comprising a solution of the compound of formula (IV) in a organic solvent, the organic solvent comprising one or more of EtOEt, MeOtBu, iPrOiPr, dioxane, cPentylOMe, THF, MeTHF, hexane, heptane, toluene, or xylene, or a mixture thereof.

Embodiment 30: The process of embodiment 29, wherein the compound of formula (IV) is in a mixture of MeTHF and toluene.

Embodiment 31: The process of embodiment 29 or embodiment 30, wherein to the solution of the compound of formula (IV) is added iPrMgBr in MeTHF.

Embodiment 32: The process of embodiment 31, wherein the molar ratio of iPrMgBr to the compound of formula (IV) is about 1:1.

Embodiment 33: The process of any one of embodiments 26-32, comprising a second CSTR comprising a mixture of the compounds of formula (IV), the Mg-imine complex in a suspension of MeTHF and toluene wherein to this mixture about 0.3 equivalents of iPrMgBr in MeTHF are added.

Embodiment 34: The process of embodiment 33, wherein the contents of the first CSTR are transferred to the second CSTR.

Embodiment 35: The process of any one of embodiments 26-34, further comprising a third CSTR, wherein the contents of the second CSTR are transferred to the third CSTR and stirred in the third CSTR.

Embodiment 36: The process of any one of embodiments 26-35, wherein the Mg-imine complex is formed as a solid in suspension by reacting the compound of formula (IV) with iPrMgBr and is transferred as a suspension between CSTRs.

Embodiment 37: The process of any one of embodiments 26-36, comprising a fourth CSTR to which aqueous NaHSO₄ is added.

Embodiment 38: The process of embodiment 37, wherein pH of a biphasic mixture after addition of the aqueous NaHSO₄ in the fourth CSTR is about 1 to about 3.

Embodiment 39: The process of embodiment 38, wherein the pH of the biphasic mixture in the fourth CSTR is about 2.

Embodiment 40: The process of any one of embodiments 26-39, wherein the contents of the third CSTR are added to the fourth CSTR, thereby forming the compound of formula (I).

Embodiment 41: The process of any one of embodiments 26-34 and claims 36-40, further comprising a fourth CSTR, wherein the contents of the second CSTR are transferred to the fourth CSTR.

Embodiment 42: The process of any one of embodiments 26-41, comprising a fifth CSTR to which a solution of a base in water is added. The base is selected from the group consisting of NaOH, KOH, Na₂CO₃, and K₂CO₃. The process of any one of embodiments 26-41, comprising a fifth CSTR to which a solution of NaOH in water is added.

Embodiment 43: The process of claim 42, wherein the pH of a biphasic mixture in the fifth CSTR after addition of NaOH is about 4 to about 7.

Embodiment 43A: The process of claim 42, wherein the pH of a biphasic mixture in the fifth CSTR after addition of NaOH is about 4 to about 5.5.

Embodiment 44: The process of claim 43, wherein the pH of the biphasic mixture in the fifth CSTR after addition of NaOH is about 4.8±0.2.

Embodiment 44A: The process of claim 43, wherein the pH of the biphasic mixture in the fifth CSTR after addition of NaOH is about 4.6±0.2.

Embodiment 45: The process of any one of embodiments 26-44, further comprising a first separation vessel.

Embodiment 46: The process of embodiment 45, wherein the contents of the fifth CSTR are transferred to the separation vessel, thereby separating organic and aqueous phases.

Embodiment 47: The process of embodiment 46, wherein the organic phase is: (a) washed with water; (b) distilled; (c) precipitated; and (d) dried to isolate the compound of formula (I).

Embodiment 48: The process of any of embodiments 26-47, further comprising a sixth CSTR to which water is added.

Embodiment 49: The process of embodiment 48, wherein the organic phase after separation is transferred to the sixth CSTR.

Embodiment 50: The process of embodiment 49, further comprising a second separation vessel.

Embodiment 51: The process of embodiment 50, wherein the contents of the sixth CSTR are transferred to the second separation vessel, thereby separating organic and aqueous phases.

Embodiment 52: The process of embodiment 51, wherein the organic phase is: (i) distilled; (ii) precipitated; and (iii) dried to isolate the compound of formula (I).

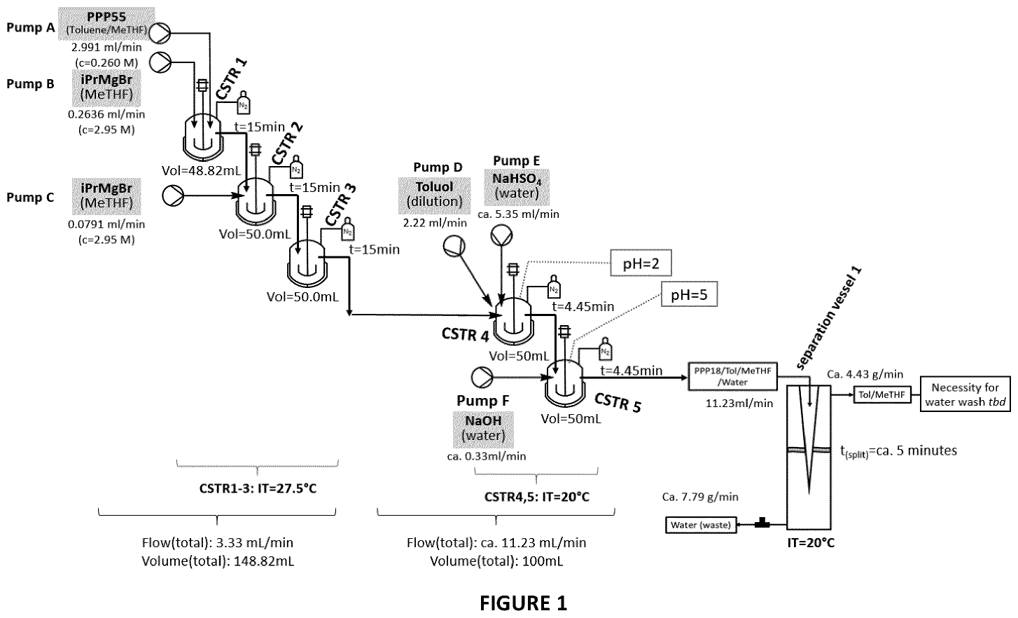

Embodiment 53: The process of any one of embodiments 1-52 substantially performed according to FIG. 1.

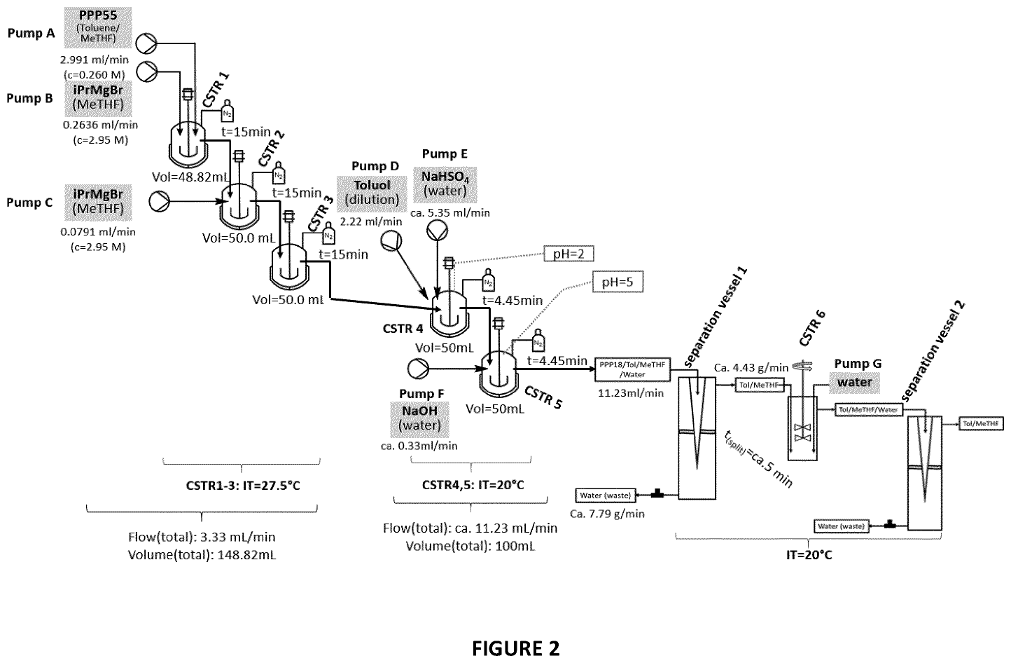

Embodiment 54: The process of any one of embodiments 1-52 substantially performed according to FIG. 2.

Embodiment 55: A process of any one of embodiments 1-54, wherein R¹ is tert-butyloxycarbonyl.

Embodiment 56A: A process for the preparation of a compound of formula (I) or a pharmaceutically acceptable salt thereof,

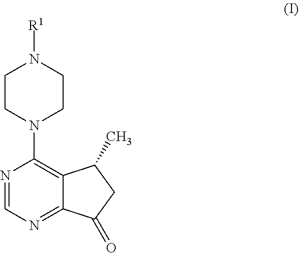

the process comprising the steps of:
(a) solubilizing a compound of formula (IV)

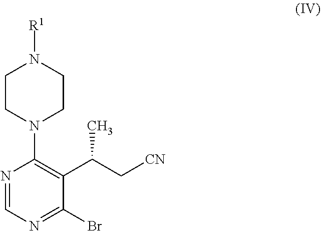

in a mixture of toluene and MeTHF in a first continuous stirred tank reactor (CSTR);
(b) adding iPrMgBr in MeTHF to the solubilized compound of step (a) in the first CSTR and, thereby forming a Mg-imine complex as a solid:
(c) transferring the contents of the first CSTR to a second CSTR to which iPrMgBr in MeTHF is added, thereby forming a Mg-imine complex as a solid;
(d) transferring the contents of the second CSTR to a third CSTR to which toluene is added, thereby forming a Mg-imine complex as a solid;
(e) transferring the contents of the third CSTR to a fourth CSTR to which aqueous NaHSO₄ is added to set a pH about 2, thereby forming the compound of formula (I) and allowing the reaction to proceed;
(f) transferring the contents of the fourth CSTR to a fifth CSTR to which NaOH in water is added to set a pH of about 5 and allowing the reaction to proceed; and
(g) transferring the contents of the fifth CSTR to a first separation vessel to separate the compound of formula (I) or a pharmaceutically acceptable salt thereof.

Embodiment 56B: A process for the preparation of a compound of formula (I) or a pharmaceutically acceptable salt thereof,

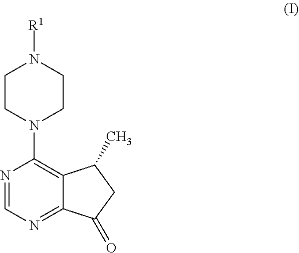

the process comprising the steps of:
(a) solubilizing a compound of formula (IV)

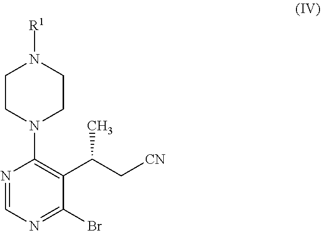

in a mixture of toluene and MeTHF in a first continuous stirred tank reactor (CSTR);
(b) adding iPrMgBr in MeTHF to the solubilized compound of step (a) in the first CSTR and, thereby forming a Mg-imine complex as a solid in suspension:
(c) transferring the contents of the first CSTR to a second CSTR to which iPrMgBr in MeTHF is added, thereby forming a Mg-imine complex as a solid in suspension;
(d) transferring the contents of the second CSTR to a third CSTR to which toluene is added, thereby forming a Mg-imine complex as a solid in suspension;
(e) transferring the contents of the third CSTR to a fourth CSTR to which aqueous NaHSO₄ is added to set a pH about 2, thereby forming the compound of formula (I) and allowing the reaction to proceed;
(f) transferring the contents of the fourth CSTR to a fifth CSTR to which NaOH in water is added to set a pH of about 5 and allowing the reaction to proceed; and
(g) transferring the contents of the fifth CSTR to a first separation vessel to separate the compound of formula (I) or a pharmaceutically acceptable salt thereof.

Embodiment 56C: A process for the preparation of a compound of formula (I) or a pharmaceutically acceptable salt thereof,

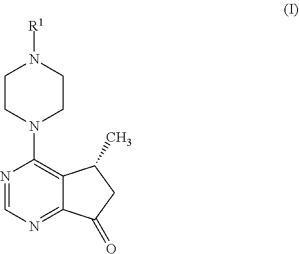

the process comprising the steps of:
(a) solubilizing a compound of formula (IV)

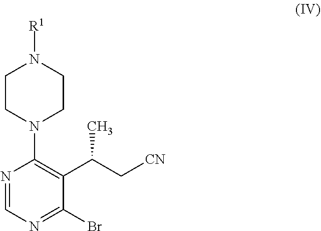

in a mixture of toluene and MeTHF in a first continuous stirred tank reactor (CSTR);
(b) adding iPrMgBr in MeTHF to the solubilized compound of step (a) in the first CSTR and wherein the first CSTR has a residence time of about 15 min, thereby forming a Mg-imine complex as a solid in suspension;
(c) transferring the contents of the first CSTR to a second CSTR to which iPrMgBr in MeTHF is added wherein the second CSTR has a residence time of about 15 min, thereby forming a Mg-imine complex as a solid in suspension;
(d) transferring the contents of the second CSTR to a third CSTR to which toluene is added wherein the third CSTR has a residence time of about 15 min, thereby forming a Mg-imine complex as a solid in suspension;
(e) transferring the contents of the third CSTR to a fourth CSTR to which aqueous NaHSO₄ is added to set a pH about 2, thereby forming the compound of formula (I) and allowing the reaction to proceed for about 4.45 min;
(f) transferring the contents of the fourth CSTR to a fifth CSTR to which NaOH in water is added to set a pH of about 5 and allowing the reaction to proceed for about 4.45 min; and
(g) transferring the contents of the fifth CSTR to a first separation vessel to separate the compound of formula (I) or a pharmaceutically acceptable salt thereof.

Embodiment 56D: Embodiment 56A: A process for the preparation of a compound of formula (I) or a pharmaceutically acceptable salt thereof,

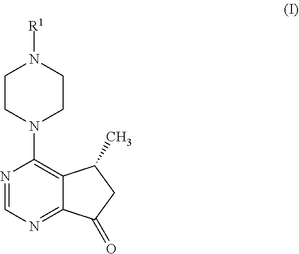

the process comprising the steps of:
(a) solubilizing a compound of formula (IV)

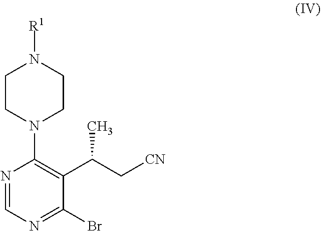

in a mixture of toluene and MeTHF in a first continuous stirred tank reactor (CSTR);
(b) adding iPrMgBr in MeTHF to the solubilized compound of step (a) in the first CSTR and, thereby forming a Mg-imine complex as a solid:
(c) transferring the contents of the first CSTR to a second CSTR to which iPrMgBr in MeTHF is added, thereby forming additional Mg-imine complex as a solid;
(d) transferring the contents of the second CSTR to a third CSTR to which toluene is added, thereby forming additional Mg-imine complex as a solid;
(e) transferring the contents of the third CSTR to a fourth CSTR to which aqueous NaHSO₄ is added to set a pH about 2, thereby forming the compound of formula (I) and allowing the reaction to proceed;
(f) transferring the contents of the fourth CSTR to a fifth CSTR to which NaOH in water is added to set a pH of about 5 and allowing the reaction to proceed; and
(g) transferring the contents of the fifth CSTR to a first separation vessel to separate the compound of formula (I) or a pharmaceutically acceptable salt thereof.

Embodiment 56E: A process for the preparation of a compound of formula (I) or a pharmaceutically acceptable salt thereof,

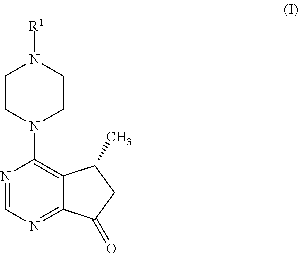

the process comprising the steps of:
(a) solubilizing a compound of formula (IV)

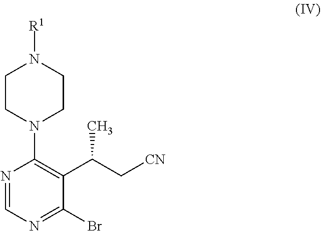

in a mixture of toluene and MeTHF in a first continuous stirred tank reactor (CSTR);
(b) adding iPrMgBr in MeTHF to the solubilized compound of step (a) in the first CSTR and wherein the first CSTR has a residence time of about 15 min, thereby forming a Mg-imine complex as a solid in suspension;
(c) transferring the contents of the first CSTR to a second CSTR to which iPrMgBr in MeTHF is added wherein the second CSTR has a residence time of about 15 min, thereby forming additional Mg-imine complex as a solid in suspension;
(d) transferring the contents of the second CSTR to a third CSTR to which toluene is added wherein the third CSTR has a residence time of about 15 min, thereby forming additional Mg-imine complex as a solid in suspension;
(e) transferring the contents of the third CSTR to a fourth CSTR to which aqueous NaHSO₄ is added to set a pH about 2, thereby forming the compound of formula (I) and allowing the reaction to proceed for about 4.45 min;
(f) transferring the contents of the fourth CSTR to a fifth CSTR to which NaOH in water is added to set a pH of about 5 and allowing the reaction to proceed for about 4.45 min; and
(g) transferring the contents of the fifth CSTR to a first separation vessel to separate the compound of formula (I) or a pharmaceutically acceptable salt thereof.

As described above the third CSTR can be skipped in any embodiment. In this case the numbering of the following CSTRs remains the same.

Embodiment 57: The process of embodiment 56, wherein the product containing organic phase is: (a) washed with water; (b) distilled; (c) precipitated; and (d) dried.

Embodiment 58: The process of embodiment 56, further comprising a sixth CSTR to which water is added.

Embodiment 59: The process of embodiment 58, wherein the organic phase after separation is transferred to the sixth CSTR.

Embodiment 60: The process of embodiment 59, further comprising a second separation vessel.

Embodiment 61: The process of claim 60, wherein the contents of the sixth CSTR are transferred to the second separation vessel, thereby separating organic and aqueous phases.

Embodiment 62: The process of embodiment 61, wherein the organic phase comprises the compound of formula (I) and is: (i) distilled; (ii) precipitated; and (iii) dried.

Embodiment 63: A process for the preparation of a compound of formula (I) or a pharmaceutically acceptable salt thereof,

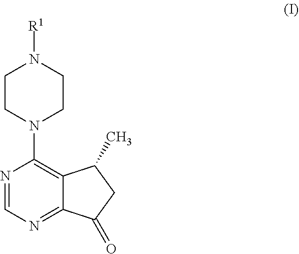

the process comprising the steps of:
(a) solubilizing a compound of formula (IV)

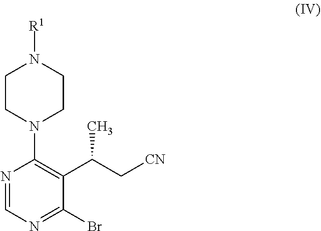

in a mixture of toluene and MeTHF;
(b) adding iPrMgBr in MeTHF to the solubilized compound of step (a) thereby forming a Mg-imine complex as a solid in suspension;
(c) adding an aqueous acid salt;
(d) adding a base in water; and
(e) isolating the compound of formula (I).

Embodiment 64: A process for the preparation of a compound of formula (I) or a pharmaceutically acceptable salt thereof,

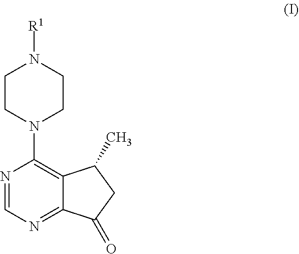

the process comprising the steps of:
(a) solubilizing a compound of formula (IV)

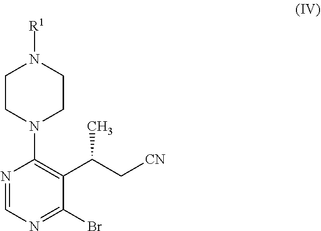

in a mixture of toluene and MeTHF;
(b) adding iPrMgBr in MeTHF to the solubilized compound of step (a) thereby forming a Mg-imine complex as a solid in suspension;
(c) adding an aqueous NaHSO₄;
(d) adding NaOH in water; and
(e) isolating the compound of formula (I).

Embodiment 65. A process for the preparation of a compound of formula (I),

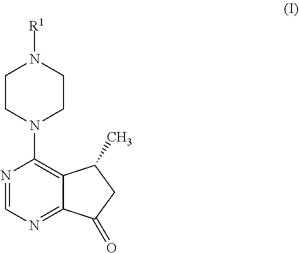

or a pharmaceutically acceptable salt thereof, wherein the process comprises a continuous flow reaction, wherein R¹ is hydrogen or an amino protecting group.

Embodiment 66. The process of embodiment 65, wherein the continuous flow reaction comprises cyclization of a compound of formula (II),

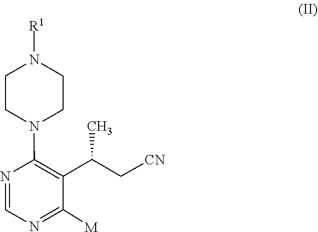

or a pharmaceutically acceptable salt thereof, wherein R¹ is hydrogen or an amino protecting group, and M is Li or Mg.

Embodiment 66a. The process of embodiment 65, wherein the continuous flow reaction comprises cyclization of a compound of formula (H),

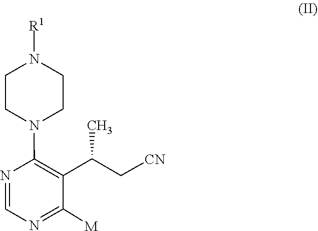

or a pharmaceutically acceptable salt thereof, wherein R¹ is an amino protecting group, and M is Li or MgX, wherein X is Br or I.

### Examples

The following Examples are presented by way of illustration, not limitation.

### Example 1

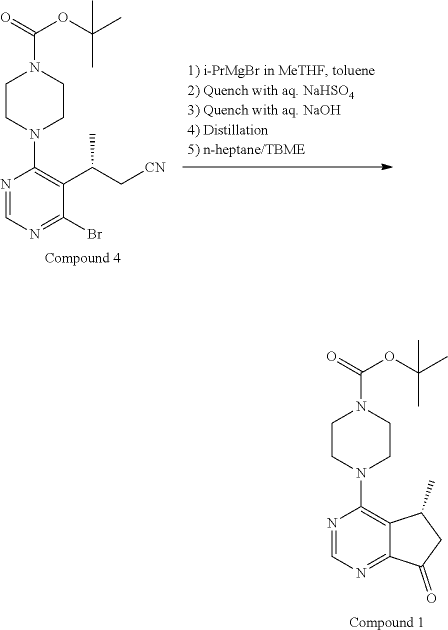

Equipment: 3×100 mL and 2×250 mL double jacketed reactor. An overflow tube (ID=3.0 mm) was positioned in each reactor to control the filling volume 50 mL. Overhead stirrers with impeller mixer were used. 4× Huber thermostats and 6× continuous Syrdos2 syringe pumps (syringe volume 1.0-10.0 mL) were used. PTFE tubing (ID=0.8 mm and 1.58 mm), 2× pH Electode InLab-Semi-Micro-L and 5× temperatures probes PT100 were used. A horizontal glass separation column (50 mL) was used for liquid/liquid phase split.

All continuous stirred tank reactors (CSTRs) were equipped with an overflow tube (ID=3 mm) to adjust the reactor volume individually. CSTR1 was adjusted to exactly 48.8 mL, whereas CSTR2-5 had a volume of 50.0 mL. The residence time in each individual CSTR is a consequence of reactor volume and flow rates as provided by the equation below.

Residence time [min]=Volume [mL]/Flow rate [mL/min]

| CSTR No | Residence Time (min) |
|---|---|
| CSTR1 | 15 |
| CSTR2 | 15 |
| CSTR3 | 15 |
| CSTR4 | 4.45 |
| CSTR5 | 4.45 |

### Preparation of Feedstock Solutions:

Feed A (tert-butyl (R)-4-(6-bromo-5-(1-cyanopropan-2-yl)pyrimidin-4-yl)piperazine-1-carboxylate (Compound 4)):

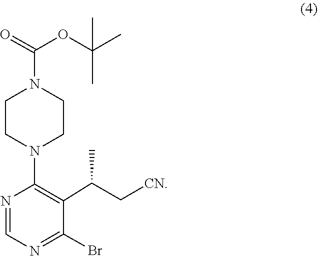 [SMILES: C[C@H](CC#N)c1c(Br)ncnc1N1CCN(C(=O)OC(C)(C)C)CC1]

Compound 4 (143.6 g) was solubilized in a mixture of toluene (772.9 g) and MeTHF (295.3 g).

The resulting brownish solution had a final concentration of 0.26 M.

Feeb B (iPrMgBr in MeTHF): iPrMgBr was supplied as a 40% (w/w) solution in MeTHF (approx 2.95 M) and used as such directly as supplied.

Feed C (aqueous NaHSO₄): NaHSO₄*H₂O (57.5 g) was solubilized in water (942.5 g). The resulting clear solution had a final concentration of 5.0% (w/w).

Feed D (aqueous NaOH): NaOH (80.0 g) was solubilized in water (990.4 g). The resulting clear solution had a final concentration of 2.0 M.

Feed E (toluene): Toluene was used as a neat solvent for dilution of the product stream.

### Example 2: Ramp-Up Phase (in Batch Mode)

All syringe pumps and transfer-lines were purged with two volumes of the according feed solution.

CSTR1 and CSTR2 were charged each with Feed A (44.9 mL). Feed B (263.6 μL, 1.00 equiv) was added into CSTR1 via dip-tube over a period of 15 minutes while maintaining an IT of 27° C. After addition, the resulting suspension showed approximately 75% conversion of Compound 4 in CSTR1. Feed B (342.7 μL, 1.30 equiv) was added into CSTR2 via dip-tube over a period of 15 minutes while maintaining an IT of 27° C. After dosage, the resulting suspension showed approximately 95% conversion of Compound 4 in CSTR2. CSTR3 was charged with toluene (15 mL) to reach minimum stirring volume and adjust to IT of 27° C. CSTR4 was charged with water (40 mL) and the pH-controlled addition of Feed C (aqueous NaHSO₄) was activated to adjust and maintain pH of 2 at an internal temperature of 15° C. CSTR5 was charged with water (40 mL) and the pH-controlled addition of Feed D (aq. NaOH) was activated to adjust and maintain pH=5 at an IT of 25° C.

### Example 3: Fully Continuous Operation

All six pumps were started simultaneously at their individual flow rates (see, for example, FIG. 1 and FIG. 2) and the reaction mass allowed to continuously overflow by gravity from CSTR to CSTR. Rates are provided below for reference.

Feed A to CSTR1: 2991 μL/min, 1.00 equiv
Feed B to CSTR1: 263.6 μL/min, 1.00 equiv
Feed B to CSTR2: 79.1 μL/min, 0.30 equiv
Feed C to CSTR4: approx 5.35 ml/min (pH
controlled dosage to maintain pH = 2)
Feed E to CSTR4: 2.22 mL/min (for dilution
of the product stream)
Feed D to CSTR5: approx 0.33 mL/min (pH
controlled dosage to maintain pH = 5)

The biphasic liquid/liquid stream exiting CSTR5 was directed to an inline settler unit to separate the two layers (see FIG. 1 and FIG. 2). The upper (organic) layer containing the desired Compound 1 was collected and the lower (aqueous) layer directed to waste.

Isolation of Compound 1 in batch-mode

The organic layer was concentrated by distillation (IT of 35° C., approx 10 mbar) to a predefined residual volume, followed by addition of an anti-solvent mixture (n-heptane:methyl-tert butylether—1:1.5 vol/vol) over 90 minutes at IT of 45° C. to complete the precipitation of Compound 1. The slurry was cooled to IT of 0° C. followed by filtration. The resulting filter cake was washed with a solvent mixture (n-heptane:methyl-tert butylether—1:1.5 vol/vol) at IT of 0° C. The product was dried at jacket temperature of 50° C. and reduced pressure until constant weight was attained. Compound 1 was isolated as a pale yellow to off-white powder in 80-85% yield.

¹H NMR (500 MHz, DMSO-cfe) δ 8.60 (s, 1H), 3.82 (m, 2H), 3.72 (m, 1H), 3.65 (m, 2H), 3.50 (m, 2H), 3.42 (m, 2H), 2.90 (dd, 1H, J=7, 19 Hz), 2.25 (dd, 1H, J=2, 19 Hz), 1.41 (s, 9H), 1.19 (d, 3H, J=7 Hz);

¹³C NMR (125 MHz, DMSO-d₆) δ 205.75, 161.96, 158.89, 157.97, 154.34, 137.39, 79.67, 45.77, 43.39, 43.25, 31.22, 28.52, 20.40; [α]₄₃₆²⁰+453.7 (c=1, MeOH);

HRMS calc'd. for C₁₇H_{24 4}O₃ [M-H]⁺: 333.1921, found: 333.1916.

All technical and scientific terms used herein have the same meaning. Efforts have been made to ensure accuracy with respect to numbers used (e.g. amounts, temperature, etc.) but some experimental errors and deviations should be accounted for.

Throughout this specification and the claims, the words “comprise,” “comprises,” and “comprising” are used in a non-exclusive sense, except where the context requires otherwise. It is understood that embodiments described herein include “consisting of” and/or “consisting essentially of” embodiments.

Where a range of values is provided, it is understood that each intervening value, to the tenth of the unit of the lower limit, unless the context clearly dictates otherwise, between the upper and lower limit of the range and any other stated or intervening value in that stated range, is encompassed herein. The upper and lower limits of these small ranges which can independently be included in the smaller rangers is also encompassed herein, subject to any specifically excluded limit in the stated range. Where the stated range includes one or both of the limits, ranges excluding either or both of those included limits are also included herein.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

1. A process for the preparation of a compound of formula (I) or a pharmaceutically acceptable salt thereof, 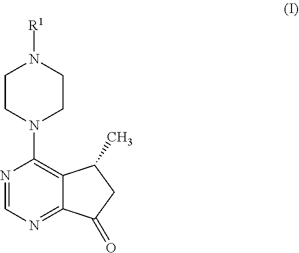
wherein the process comprises a continuous flow reaction, which continuous flow reaction comprises cyclization of a compound of formula (II): 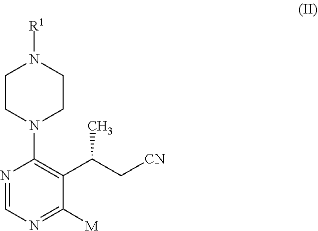
or a pharmaceutically acceptable salt thereof,
wherein:
R¹ is an amino protecting group; and
M is MgX wherein X is Br.

2. The process according to claim 1, wherein the continuous flow reaction comprises:
(a) contacting a compound of formula (III) or a pharmaceutically acceptable salt thereof 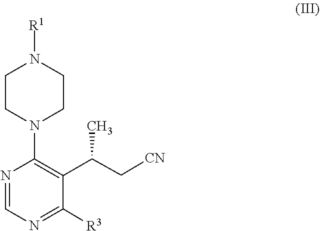
wherein:
R¹ is an amino protecting group; and
R³ is bromo;
with a Mg metalating agent to form a compound of formula (I).

3. The process of claim 2, wherein the compound of formula (III) is a compound of formula (IV) or a pharmaceutically acceptable salt thereof 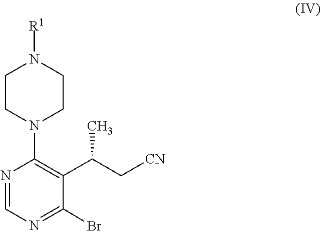
wherein R¹ is an amino protecting group.

4. The process of claim 3, wherein the metalating agent is iPrMgBr.

5. The process of any one of claims 1-4, wherein said continuous flow reaction is conducted in a coordinating ethereal solvent or a non-coordinating apolar solvent, or a mixture thereof.

6. The process of any one of claims 1-5, wherein said continuous flow reaction is conducted in a coordinating ethereal solvent.

7. The process of any one of claims 1-5, wherein said continuous flow reaction is conducted in a non-coordinating apolar solvent.

8. The process of claim 6, wherein the coordinating ethereal solvent is EtOEt, MeOtBu, iPrOiPr, dioxane, cPentylOMe, THF, MeTHF or a mixture thereof.

9. The process of claim 7, wherein the non-coordinating apolar solvent is hexane, heptane, toluene, or xylene, or a mixture thereof.

10. The process of any one of claims 4-9, wherein the iPrMgBr is in MeTHF.

11. The process of any one of claims 3-10, wherein the compound of formula (IV) is in EtOEt, MeOtBu, iPrOiPr, dioxane, cPentylOMe, THF, MeTHF, hexane, heptane, toluene, or xylene, or a mixture thereof.

12. The process of claim 13, wherein the compound of formula (IV) is in a mixture comprising MeTHF and toluene.

13. The process of any one of claims 4-12, wherein the iPrMgBr is added to the compound of formula (IV).

14. The process of any one of claims 2-13, wherein in step (a) the compound of formula (III) is transferred to the compound of formula (II) and the compound of formula (II) is transferred to a Mg-imine complex;
and R¹ is hydrogen or an amino protecting group and wherein in in step (b) an aqueous acid salt is added to the mixture obtained from step (a), thereby forming the compound of formula (I) or a pharmaceutically acceptable salt thereof.

15. The process of claim 14, wherein the Mg-imine complex is a solid in suspension.

16. A process for the preparation of a compound of formula (I) or a pharmaceutically acceptable salt thereof, 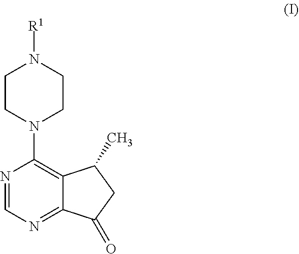
the process comprising the steps of:
(a) solubilizing a compound of formula (IV) 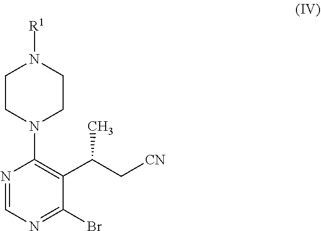
in a mixture of toluene and MeTHF;
(b) adding iPrMgBr in MeTHF to the solubilized compound of step (a) thereby forming a Mg-imine complex as a solid in suspension;
(c) adding an aqueous acid salt;
(d) adding a base in water; and
(e) isolating the compound of formula (I).

17. The process of any one of claims 4-16, wherein the molar ratio of iPrMgBr to compound of formula (IV) is about 1:1 to about 1.5:1.

18. The process of claim 7, wherein the molar ratio of iPrMgBr to compound of formula (IV) is about 1.3 to about 1.

19. The process of claim 17 or claim 18, wherein the molar ratio of iPrMgBr to compound of formula (IV) is about 1:1.

20. The process of any one of claims 4-19, wherein the iPrMgBr is added to the compound of formula (IV) in one or more steps.

21. The process of any one of claims 14-20, wherein the aqueous acid salt is selected from the group consisting of acetic acid, HCl, H₂SO₄, NaHSO₄, H₃PO₄, NaH₂PO₄, Na₂HPO₄, NaH₂Citrate, NH₄Cl, or oxalic acid, or a mixture thereof.

22. The process of claim 21, wherein the aqueous acid salt is NaHSO₄.

23. The process of any one of claims 1-22, wherein the compound of formula (I) is optionally (a) washed; (b) distilled; (c) precipitated; and (d) dried.

24. The process of any one of claims 1-23, wherein the process does not require a re-crystallization step.

25. The process of any one of claims 1-24, wherein the process is performed in one or more continuous stirred tank reactors (CSTR).

26. The process of claim 25, wherein the process is performed using 1, 2, 3, 4, 5, 6, or 7 CSTRs.

27. The process of claim 25 or claim 26, wherein the process is performed using 5 CSTRs.

28. The process of any one of claims 25-27, comprising a first CSTR containing a solution of the compound of formula (IV) in an organic solvent, the organic solvent comprising one or more components such as EtOEt, MeOtBu, iPrOiPr, dioxane, cPentylOMe, THF, MeTHF, hexane, heptane, toluene, or xylene, or a mixture thereof.

29. The process of claim 28, wherein the compound of formula (IV) is in a mixture of MeTHF and toluene.

30. The process of claim 28 or claim 29, wherein to the solution of the compound of formula (IV) in the first CSTR is added iPrMgBr in MeTHF.

31. The process of claim 30 wherein the molar ratio of iPrMgBr to the compound of formula (IV) is about 1:1.

32. The process of any one of claims 25-31, comprising a second CSTR comprising a mixture of the compounds of formula (IV) and the Mg-imine complex in a suspension of MeTHF and toluene wherein to this mixture about 0.3 equivalents of iPrMgBr in MeTHF are added.

33. The process of claim 32, wherein the contents of the first CSTR are transferred to the second CSTR.

34. The process of any one of claims 25-33, further comprising a third CSTR, wherein the contents of the second CSTR are transferred to the third CSTR and stirred in the third CSTR.

35. The process of any one of claims 25-34, wherein the Mg-imine complex is formed as a solid in suspension by reacting the compound of formula (IV) with iPrMgBr and is transferred as a suspension between CSTRs.

36. The process of any one of claims 25-35, comprising a fourth CSTR to which aqueous NaHSO₄ is added.

37. The process of claim 36, wherein pH of a biphasic mixture after addition of the aqueous NaHSO₄ in the fourth CSTR is about 1 to about 3.

38. The process of claim 37 wherein the pH of the biphasic mixture in the fourth CSTR is about 2.

39. The process of any one of claims 25-38, wherein the contents of the third CSTR are added to the fourth CSTR, thereby forming the compound of formula (I).

40. The process of any one of claims 25-33 and claims 35-39, further comprising a fourth CSTR, wherein the contents of the second CSTR are transferred to the fourth CSTR.

41. The process of any one of claims 25-40, comprising a fifth CSTR to which a solution of a base in water is added.

42. The process of claim 41, where as a base in water NaOH in water is added.

43. The process of claim 42, wherein the pH of a biphasic mixture in the fifth CSTR after addition of NaOH is about 4 to about 7.

44. The process of claim 43, wherein the pH of the biphasic mixture in the fifth CSTR after addition of NaOH is about 4.6±0.2.

45. The process of any one of claims 25-44 further comprising a first separation vessel.

46. The process of claim 46, wherein the contents of the fifth CSTR are transferred to the first separation vessel, thereby separating organic and aqueous phases.

47. The process of claim 46, wherein the product containing organic phase is: (a) washed with water; (b) distilled; (c) precipitated; and (d) dried to isolate the compound of formula (I).

48. The process of any of claims 25-47, further comprising a sixth CSTR to which water is added.

49. The process of claim 48, wherein the organic phase after separation is transferred to the sixth CSTR.

50. The process of claim 49, further comprising a second separation vessel.

51. The process of claim 50, wherein the contents of the sixth CSTR are transferred to the second separation vessel, thereby separating organic and aqueous phases.

52. The process of claim 51, wherein the organic phase is: (i) distilled; (ii) precipitated;
and (iii) dried to isolate the compound of formula (I).

53. The process of any one of claims 1-52 substantially performed according to FIG. 1.

54. The process of any one of claims 1-52 substantially performed according to FIG. 2.

55. A process of any one of claims 1-54, wherein R¹ is tert-butyloxycarbonyl.

56. A process for the preparation of a compound of formula (I) or a pharmaceutically acceptable salt thereof, 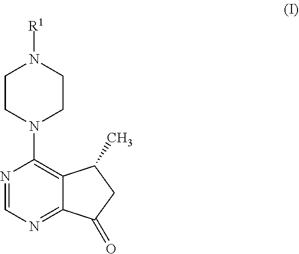
the process comprising the steps of:
(a) solubilizing a compound of formula (IV) 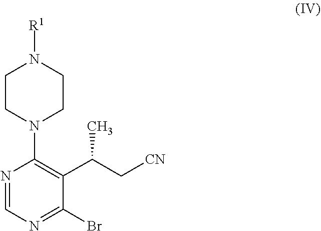
in a mixture of toluene and MeTHF in a first continuous stirred tank reactor (CSTR);
(b) adding iPrMgBr in MeTHF to the solubilized compound of step (a) in the first CSTR, thereby forming a Mg-imine complex as a solid;
(c) transferring the contents of the first CSTR to a second CSTR to which iPrMgBr in MeTHF is added, thereby forming a Mg-imine complex as a solid;
(d) transferring the contents of the second CSTR to a third CSTR to which toluene is added, thereby forming a Mg-imine complex as a solid;
(e) transferring the contents of the third CSTR to a fourth CSTR to which aqueous NaHSO₄ is added to set a pH about 2, thereby forming the compound of formula (I) and allowing the reaction to proceed;
(f) transferring the contents of the fourth CSTR to a fifth CSTR to which NaOH in water is added to set a pH of about 5 and allowing the reaction to proceed; and
(g) transferring the contents of the fifth CSTR to a first separation vessel to separate the compound of formula (I) in the organic phase.

57. A process for the preparation of a compound of formula (I) or a pharmaceutically acceptable salt thereof, 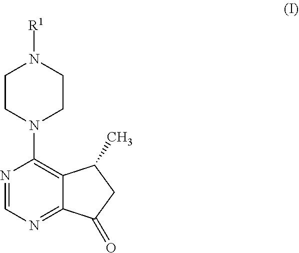
the process comprising the steps of:
(a) solubilizing a compound of formula (IV) 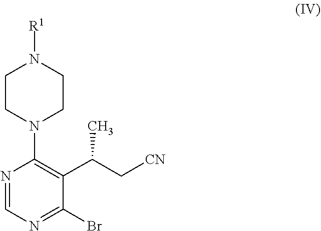
in a mixture of toluene and MeTHF in a first continuous stirred tank reactor (CSTR);
(b) adding iPrMgBr in MeTHF to the solubilized compound of step (a) in the first CSTR and wherein the first CSTR has a residence time of about 15 min, thereby forming a Mg-imine complex as a solid;
(c) transferring the contents of the first CSTR to a second CSTR to which iPrMgBr in MeTHF is added wherein the second CSTR has a residence time of about 15 min, thereby forming a Mg-imine complex as a solid;
(d) transferring the contents of the second CSTR to a third CSTR to which toluene is added wherein the third CSTR has a residence time of about 15 min, thereby forming a Mg-imine complex as a solid;
(e) transferring the contents of the third CSTR to a fourth CSTR to which aqueous NaHSO₄ is added to set a pH about 2, thereby forming the compound of formula (I) and allowing the reaction to proceed for about 4.45 min;
(f) transferring the contents of the fourth CSTR to a fifth CSTR to which NaOH in water is added to set a pH of about 5 and allowing the reaction to proceed for about 4.45 min; and
(g) transferring the contents of the fifth CSTR to a first separation vessel to separate the compound of formula (I) in the organic phase.

58. The process of claim 56 or claim 57, wherein the Mg-imine complex is formed as solid in suspension.

59. The process of any one of claims 56 to 58, wherein the product containing organic phase is: (i) washed with water; (ii) distilled; (iii) precipitated; and (iv) dried.

60. The process of claim 58, further comprising a sixth CSTR to which water is added.

61. The process of claim 60, wherein the organic phase after separation is transferred to the sixth CSTR.

62. The process of claim 61, further comprising a second separation vessel.

63. The process of claim 62, wherein the contents of the sixth CSTR are transferred to the second separation vessel, thereby separating organic and aqueous phases.

64. The process of claim 63, wherein the organic phase is: (i) distilled; (ii) precipitated; and (iii) dried.
